(19) United States
(12) Patent Application Publication
(10) Pub. No.: US 2008/0220537 A1
(43) Pub. Date: Sep. 11, 2008

(54) Substrates and methods for selective immobilization of active molecules

(75) Inventor: Mathieu Foquet, Redwood City, CA (US)

Correspondence Address: Pacific Biosciences of California, Inc., 1505 Adams Drive, Menlo Park, CA, 94025, US

(73) Assignee: Pacific Biosciences of California, Inc., Menlo Park, CA (US)

(21) Appl. No.: 12/074,716

(22) Filed: Mar. 5, 2008

Related U.S. Application Data
(60) Provisional application No. 60/905,786, filed on Mar. 7, 2007.

(51) Int. Cl. G01N 33/543

(52) U.S. Cl. 436/518

(57) ABSTRACT

Substrates and methods for providing increased selectivity in the immobilization of active molecules of interest in desired locations of substrates for use in analytical operations and particularly optical analytical operations.

### CROSS-REFERENCE TO RELATED APPLICATIONS

This application claims priority from Provisional U.S. Pat. No. 60/905,786, filed Mar. 7, 2007, the full disclosure of which is incorporated herein by reference in its entirety for all purposes.

### STATEMENT REGARDING FEDERALLY SPONSORED RESEARCH

Not applicable

### BACKGROUND OF THE INVENTION

There are a wide range of analytical operations that may be benefited from the ability to analyze the reaction of individual molecules, relatively small numbers of molecules or molecules at relatively low concentrations. A number of approaches have been described for providing these sparsely populated reaction mixtures. For example, in the field of nucleic acid sequence determination, a number of researchers have proposed single molecule, or low concentration approaches to obtaining sequence information in conjunction with the template dependent synthesis of nucleic acids by the action of polymerase enzymes.

The various different approaches to these sequencing technologies offer different methods of monitoring only one or a few synthesis reactions at a time. For example, in some cases, the reaction mixture is apportioned into droplets that include low concentrations of reactants. In other applications, certain reagents are immobilized onto surfaces such that they may be monitored without interference from other reaction components in solution. In still another approach, optical confinement techniques are used to ascertain signal information only from a relatively small number of reactions, e.g., a single molecule, within an optically confined area. Notwithstanding the availability of the above-described techniques, there are instances where further selectivity of reaction components for analysis would be desirable. The present invention meets these and a variety of needs.

### BRIEF SUMMARY OF THE INVENTION

The present invention is directed to methods and substrates that have enhanced selectivity in the immobilization of active molecules in desired locations. In particularly preferred aspects, the substrates and methods of the invention employ fabrication materials and methods that result in areas that have enhanced or decreased ability to bind or be bound to active molecules of interest, where such selectivity may be applied in either or both of selectivity in binding and/or selectivity in activation or deactivation of molecules already bound.

### BRIEF DESCRIPTION OF THE DRAWINGS

FIG. 1 schematically illustrates a substrate having active molecules of interest localized in a desired location on such substrates.

FIG. 2 schematically illustrates a first aspect of the invention that provides for enhanced selective localization of active molecules of interest in desired locations of a substrate.

FIG. 3 schematically illustrates an alternative aspect of the invention in which active molecules of interest are localized in desired locations of a substrate.

FIG. 4 schematically illustrates the use of substrates as shown in FIG. 2, in optical analytical operations, where the desired locations include optical confinements.

FIG. 5 schematically illustrates the use of substrates as shown in FIG. 3, in optical analytical operations, where the desired locations include optical confinements.

FIG. 6 illustrates a further alternative layered substrate structure in accordance with the invention.

### DETAILED DESCRIPTION OF THE INVENTION

The present invention is generally directed to substrates having molecules of interest disposed upon portions of the substrate in certain desired locations, and consequently, not disposed in other, less desired or undesired locations. In particular, the present invention employs substrates comprised of different material regions that provide for increased or decreased binding, activity and/or viability of molecules of interest, when immobilized thereto, relative to other regions of the substrate. In particular, the substrates of the invention provide differential surfaces that have, at different, selected regions, chemical or physical properties that either (1) substantially prevent or inhibit binding of active molecules of interest to such regions, or degrade or otherwise deactivate molecules of interest in such regions, or conversely (2) substantially enhance binding or activity of molecules of interest in such regions relative to the other regions on the substrate.

The substrates of the invention are generally useful in performing analytical chemical and/or biochemical reactions in a planar array format and particularly those that are monitored using optical signaling components within the reaction of interest. As a result, the substrates typically have a planar construction and include an optical access window in order to obtain signals from the region of the substrate in which such reactions are occurring.

In particularly preferred aspects, the differential properties of the different regions of the substrate are provided during underlying fabrication of the solid substrate upon which the molecules of interest are to be disposed, and are comprised of different layered elements of the overall substrate. In particular, the invention provides layered substrates that include a first layer having a first surface and at least a second layer disposed on a portion of the first surface, such that the second layer provides substantially different ability to have disposed thereon or bound thereto, active molecules of interest, relative to the first surface of the first substrate.

In the context of the invention, the phrase “substantially increased or decreased ability to bind active molecules” of interest to a given region encompasses situations where the number of molecules bound to the surface is substantially increased or decreased relative to other regions of the substrate, and thus resulting in substantially different numbers of active molecules so bound. This is also referred to herein as having molecules of interest preferentially bound to one region over another. Likewise, the phrase encompasses situations where the number of molecules bound to one region relative to another is unchanged, but where molecules in one region are substantially more or less active in their desired application than molecules in the other region. In such a situation, the surface is also referred to as being an activating or deactivating surface.

In addition to surfaces that provide for enhanced or decreased activity of bound molecules of interest, the phrase “substantially increased or decreased ability to bind” also can refer to the relative ability of a first selected surface region to bind such active molecules, as described above, as compared to a second selected region on the overall surface of the substrate. In terms of the invention, a substantial increase or decrease in ability to bind refers to an increase or decrease in the number of active molecules of interest bound to the selected region of at least 10% relative to the other compared region. In preferred aspects, the increase or decrease is at least 20%, more preferably at least 50%, and still more preferably, at least 90% or more. Restated in terms of relative binding, it will be understood that a substantial increase in ability of one region to bind active molecules of interest relative to another region will mean that active molecules of interest will be preferentially bound to the first region as compared to the second region at a ratio of at least 2:1, preferably, at least 5:10, more preferably at least 10:1, and in some cases 50:1 or even 100:1 or greater. Thus, in the case where the ratio is 5:1, it will be understood that the ratio of the density of bound molecules in a first surface area will five times greater than the binding density in the second area. In contrast, a substantially decreased ability to bind active molecules of interest from one region to another will have the inverse ratios, e.g., 1:2, 1:5, 1:10, etc. Thus, in the case of the 1:5 ratio, the region having a decreased ability to bind active molecules of interest will have a density of active molecules that is one fifth that of the other region to which it is being compared.

As noted, the layered construction of the substrates, in preferred aspects, provides for the differential binding described herein. In particular, the substrates of the invention include different layers having exposed surface portions, where the exposed surface portions of the different substrates have substantially different abilities to bind the active molecules of interest. Although a variety of different materials may be employed to provide increased or decreased ability to bind active molecules of interest in accordance with the invention, in particularly preferred aspects, the layers of the devices described herein that provide such differential abilities will comprise one or more metals and/or semiconductors that are deposited upon the substrates of the invention.

In a first example, a first substrate layer may be comprised of a material that is readily compatible with the immobilization of active molecules of interest. In addition, the underlying substrate layer also will typically permit optical access to reactions being carried out on the surface of the substrate. Accordingly, transparent substrates are particularly preferred. While transparent polymeric substrates, such as polymethylmethacrylate (PMMA), polystyrene, polycarbonate substrates and the like, may be used, silica based substrates, such as glass, quartz, fused silica, silicon, silicon nitride, silicate substrates are preferred. In such cases, and for the purposes described elsewhere herein, a second layer may be provided over the first, leaving regions of the surface of the first substrate exposed so as to provide optically accessible regions of the substrate surface.

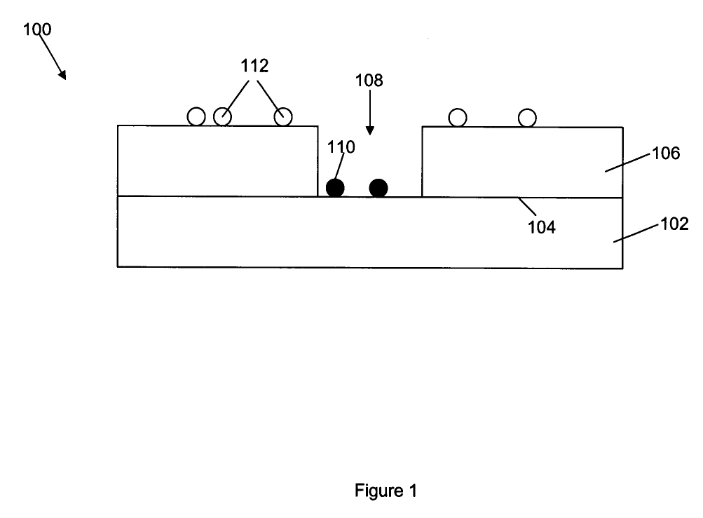

The second layer is provided having a decreased ability to bind active molecules of interest, e.g., a deactivating surface, relative to the surface of the first layer. As a result, the likelihood will be greater that active molecules of interest will be substantially immobilized only on the exposed portions of the first layer surface, and not in other regions covered by the second layer. FIG. 1 provides a schematic illustration of this aspect of the invention. As shown, an overall substrate 100 includes a transparent or other first layer 102, having a first surface 104. A second layer 106 is disposed over a portion of the first surface, leaving other portion(s) of the first surface, e.g., region 108, exposed. In the context of the present invention, the second layer 106 will have an increased or decreased ability to bind to active molecules of interest. As shown, the second layer is shown as having a decreased ability to bind active molecules by virtue of its deactivating influence on any molecules bound thereto. This is schematically illustrated as active molecules of interest 110 (filled circles) that are located on the first surface 104 of the first layer 102, while molecules of interest 112 bound to the surface of the second layer 106 are inactive (illustrated as open circles).

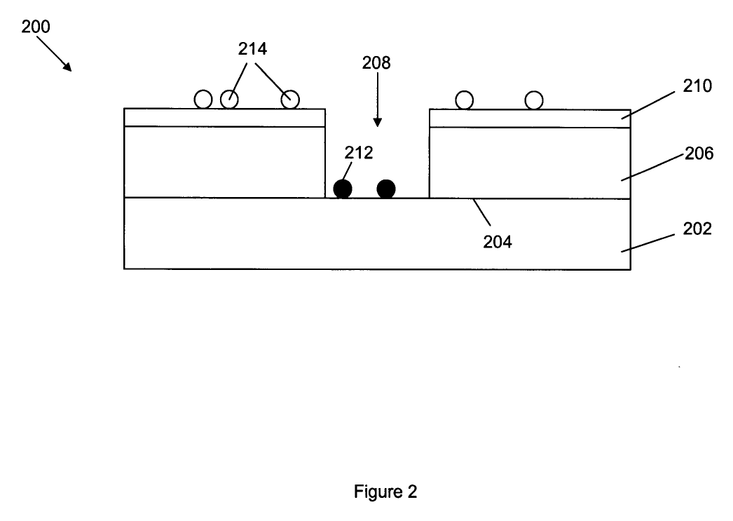

The layered substrates of the invention may also include more than two layers, as shown in FIG. 1. In particular, in some cases, multilayered structures be used to provide additional functionalities to the overall substrate, or to provide further variability in the immobilization of the active molecules of interest. For example, in some cases, one may apply an activating or deactivating layer as a final layer in the construction of a particular substrate, in order to provide for selective immobilization within more complex devices. For example, as shown in FIG. 2, an overall substrate 200 may comprise a first transparent layer 202 having a second layer 206 deposited over a portion of its surface 204. Openings are provided through the second layer 206, such as zero mode waveguide core 208, so as to provide optical accessibility to reactions occurring at or near the surface of transparent substrate 202. A third or subsequent layer 210 is deposited over the top of layer 206, which third layer provides for increased or decreased ability to bind active molecules of interest. As shown, the third layer 208 provides a deactivating influence on the molecules of interest 214 (shown deactivated as open circles) while active molecules of interest 212 (shown as filled circles) are immobilized on the surface of transparent substrate 202.

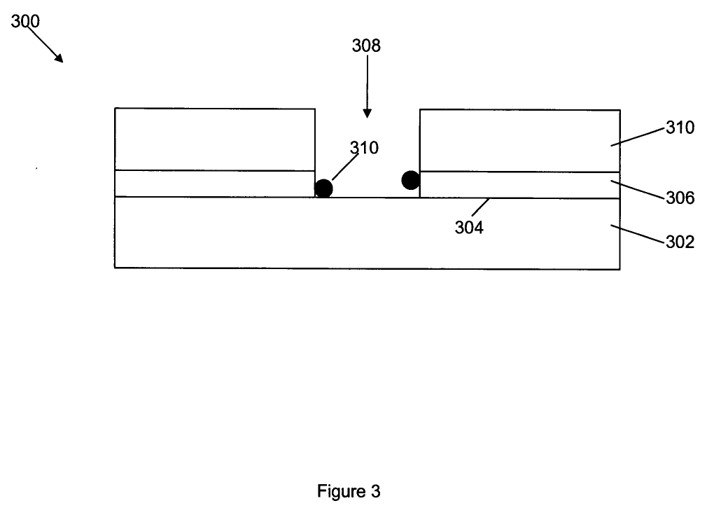

Although described above in terms of providing an activating or deactivating layer as the final constructed layer on a substrate, as alluded to above, one may wish to use intermediate layers in the modulation of relative immobilization of molecules of interest to the substrates if the invention. For example, in some cases, it may be desirable to provide isolated surface regions having enhanced ability to bind molecules of interest relative to the rest of the surface of the substrate. An example of this is shown in FIG. 3. As shown, an overall substrate 300 again includes a first transparent substrate layer 302 having a first surface 304. A second layer 306 is again deposited upon a portion of the surface 304 of substrate 302, leaving openings 308 to the transparent substrate 302 surface 304. The second layer 306 provides for an increased or enhanced ability to bind active molecules of interest relative to at least one of the underlying transparent substrate 302 or the third or subsequent layer 310 that is provided over the second layer 306. As a result, active molecules of interest 312 are selectively bound to the exposed portions 314 of layer 306 at or near the bottom of the opening 308, and are consequently optically accessible for detection systems placed below the transparent substrate 302.

In the context of the present invention, a variety of different materials may be employed that will have decreased ability to have active molecules of interest immobilized thereon. In some cases, such materials may provide a deactivating influence over the molecules of interest. For example, TiO₂ layers may be provided that, upon exposure to appropriate wavelength light, e.g., light in the UV range, will oxidize organic materials, including certain molecules of interest, e.g., proteins, enzymes, or other organic materials. Other materials that can provide deactivating influences include, for example, deactivating enzymes, like proteases and DNase, as well as surface bound surfactants, and the like. In contrast to those surface materials that operate to deactivate molecules of interest that are bound thereto, other surface materials may simply be less likely to bind those molecules. For example, in certain aspects, more inert surfaces may be used as having reduced ability to bind the active molecules of interest. By way of example, noble metals, such as platinum may be used, that are non-reactive (and consequently non-binding) with the active molecules of interest. Similarly, less reactive dielectric materials may be employed as the nonbinding surface, such as silicon nitride (SiN) and titanium nitride (TiN).

Other materials may be provided that have a substantially reduced ability to bind to the molecules of interest. For example, in the case of molecules of interest that carry a net negative or positive charge, an oppositely charged material may be employed to reduce the amount of association between such material and the molecules of interest. Such an approach is particularly useful in cases where the molecules of interest are highly charged.

The above described substrates are particularly useful in analytical operations where it is desirable to provide and monitor active molecules of interest in only selected regions of a substrate. For example, in the case of enzyme driven analytical operations, the substrates of the invention can provide for selective, concentrated immobilization of the subject enzyme within an observation region on the substrate, while the additional layers have reduced or irrelevant levels of the enzyme immobilized thereon, that might contribute detrimentally to the analysis.

Such enzyme driven analyses may include any of a variety of industrially relevant enzyme systems, including those used in pharmaceutical research and discovery, such as proteases, phosphatases, kinases, nucleases, polymerases, and the like, as well as those used in more industrial applications, such as amylases, cellulases, lipases, and the like. In particularly preferred aspects, the substrates of the invention are used in the performance of nucleic acid sequence determination through the monitoring of polymerase mediated, template dependent DNA synthesis. In such cases, it is generally desirable to provide a polymerase enzyme immobilized within an observation region of a substrate, but not upon other regions (See, e.g., copending published U.S. Patent Application No. 2007/0238679, filed Mar. 30, 2006 and U.S. Pat. Nos. 7,056,661, 7,052,847, 7,033,764 and 7,056,676. the full disclosures of each of these being incorporated herein by reference in their entirety for all purposes). In particularly preferred aspects, the substrate includes optically confined regions on the surface of a substrate in which the molecules of interest are selectively immobilized. Examples of such regions include regions immediately above exposed waveguides within the underlying substrate (See, e.g., copending U.S. patent application Ser. No. 60/841,897, filed Sep. 1, 2006 and incorporated herein by reference in its entirety for all purposes), or they may include optical confinements built upon the surface of the substrate, such as one or more zero mode waveguides (See, e.g., U.S. Pat. Nos. 6,991,726 and 7,013,054 each of which is hereby incorporated herein by reference in its entirety for all purposes).

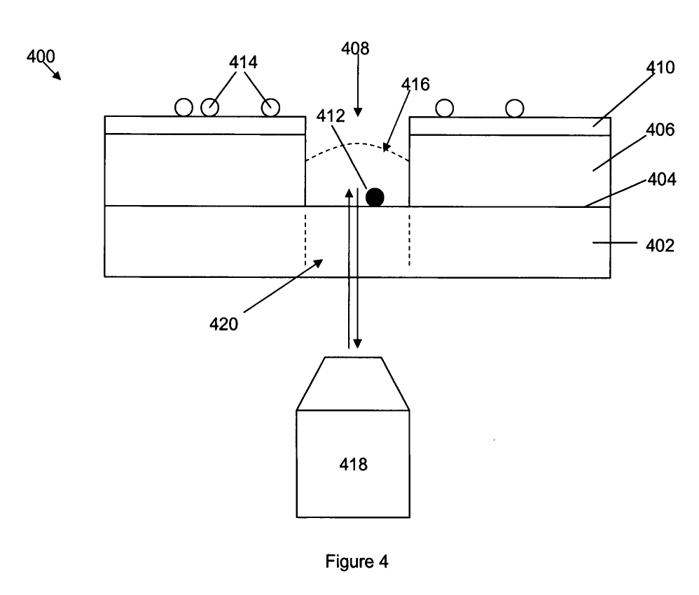

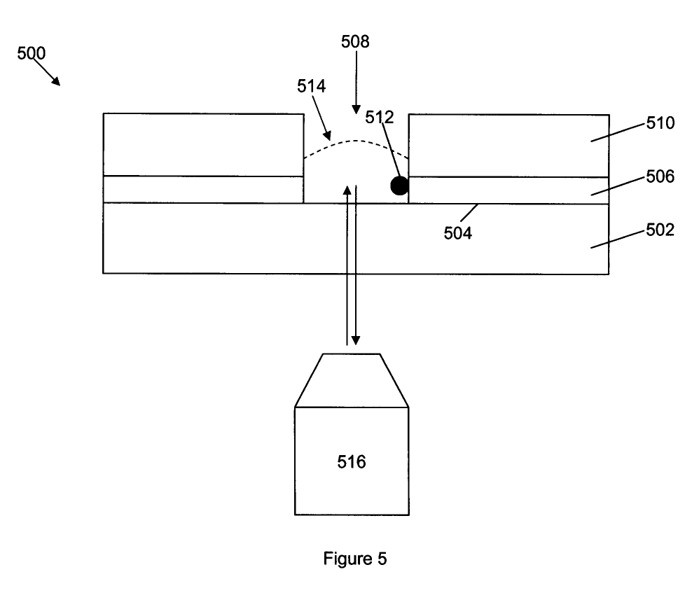

An example of the application of the present invention to optically confined regions of a substrate, is illustrated in FIGS. 4 and 5. As shown in FIG. 4, an overall substrate 400 includes transparent substrate layer 402, which is provided having a cladding or confining layer 406 disposed over the first surface 404 of the transparent substrate layer 402. The cladding layer is only provided over a portion of the transparent substrate layer 402, so as to leave observation regions 408 on the overall substrate in which the molecules of interest may be immobilized. In certain preferred embodiments, these openings 408 comprise zero mode waveguides, where the cross sectional dimension of the opening 408 is sufficiently small so as to prevent propagation of light through the waveguide. The upper surface of layer 406 is provided having a reduced ability to bind active molecules of interest, e.g., is deactivating to polymerase molecules. While the second layer may be solely comprised of an appropriate material, in some cases and as shown, an additional layer 410 is disposed over an intermediate layer 406 to provide a deactivating surface to polymerase molecules 414. The resulting substrate having active polymerase molecules 412 immobilized substantially only within the observation region, is then used to monitor the activity of the polymerase enzyme, e.g., in template mediated primer extension reactions, as detected by a detection system 418 positioned below the transparent substrate 402, such that the observation region 408 includes an observation window or aperture 420 through the transparent substrate. Where the opening 408 comprises an optically confined space, e.g., a zero mode waveguide, only a portion of the volume of the opening 508 is exposed to illumination. This is schematically illustrated by dashed line 416.

In contrast to the foregoing, and as schematically illustrated with respect to an overall substrate 500 in FIG. 5, a layer 506 may be provided over the underlying transparent substrate 502, that includes an increased ability to bind active molecules of interest, e.g., polymerase enzymes. In particular, a first transparent substrate layer 502 has provided upon its surface 504, a second layer 506 that leaves portions 508 of the underlying substrate layer 502 open to provide optical access. A third layer 510 is deposited over the second layer 506 so as to allow portions 508 to remain open as optical access. The second layer 506 comprises a material that has an increased ability to bind the molecules of interest, e.g., polymerase enzymes 512, relative to both the first transparent substrate layer 502 and the third layer 510. As a result, the molecules of interest 512 are selectively and preferentially immobilized upon the exposed portions of second layer 502. In the context of a zero mode waveguide structure, this permits immobilization of molecules of interest at a specific location in the structure of the waveguide core, e.g., at or near the bottom surface of the core, and within the observation or illumination region, the boundary of which is illustrated by the dashed line 514.

As noted above, in particularly preferred aspects, the differential binding layers employed in the substrates of the invention will comprise metal or semiconductor materials. Deposition of metal layers may generally be carried out by a variety of known methods, including sputtering, evaporation atomic layer deposition (ALD) and/or chemical vapor deposition (CVD) methods that are well known in the art. Likewise, where semiconductor materials are being deposited over underlying substrate layers, any of a variety of known methods may be employed to accomplish this, including, again, CVD methods such as plasma enhanced CVD (peCVD) and ALD methods.

By way of example, where metal or semiconductor layers are being applied over a base substrate layer, but where it is desired to retain apertures or openings in such layers to the underlying transparent substrate (e.g., zero mode waveguide cores), one can readily use the fabrication methods described in commonly owned U.S. Pat. No. 7,170,050, which is incorporated herein by reference in its entirety for all purposes. In brief, a resist layer is deposited over a transparent substrate and a series of posts are developed from the resist layer, e.g., using e-beam exposure and development, where the posts define the negative of the openings. The metal or other layer is then deposited over the underlying substrate and posts. Removal of the posts, e.g., using a liftoff or knock off process, then yields openings defined through the newly added layer. As will be appreciated, multiple different layers may be deposited over the substrate and posts to define the stratified substrate construction of certain aspects of the invention, e.g., as in FIGS. 2 and 3. For example, with respect to the substrate illustrated in FIG. 2, the desired material 210 having an increased or decreased ability to bind active molecules of interest is deposited over an intermediate layer 206 (e.g., that acts as a cladding layer for a zero mode waveguide structure). In contrast, and as illustrated in FIG. 3, the intermediate layer 306 possesses the increased or decreased ability to bind active molecules of interest, while the additional layer 310 provides an additional cladding layer for the device. As will be appreciated, when using the process outlined herein, it may be desirable to employ an anisotropic deposition process to provide the intermediate layers, e.g. rather than a conformal or isotropic process. In particular, it will generally be desirable to only deposit the intermediate material layer upon the surface of the substrate and not upon the sides of the pillars, as such would result in creation of a sleeve of the intermediate material within the aperture. As will be appreciated, in some instances, such a sleeve may be a desirable feature. However, where it is desired to maintain the intermediate material only at the bottom end of the aperture, anisotropic processes are preferred. Anisotropic processes can include a variety of processes known in the art, including sputtering, thermal or e-beam evaporation processes, and the like.

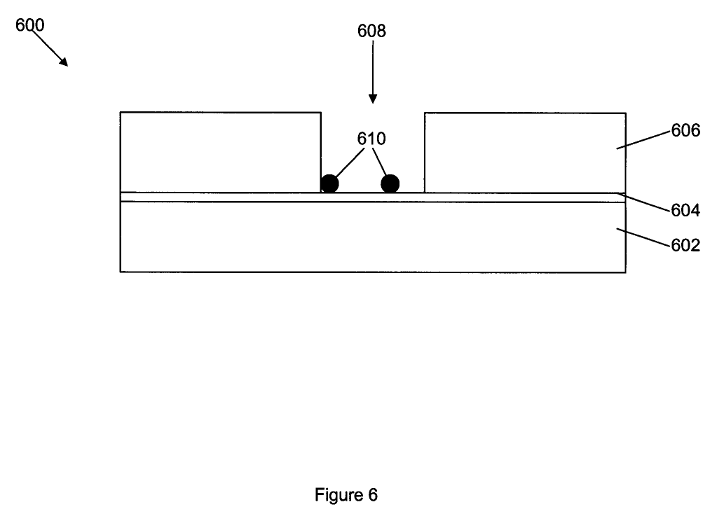

In a further aspect, illustrated in FIG. 6, one may avoid more complex deposition strategies for the intermediate layer by providing that layer prior to the other fabrication processes, including deposition, exposure and developing of resist layers. As shown, an overall substrate 600 includes a base layer such as transparent layer 602. A first intermediate layer 604 is deposited upon the base layer 602. For use in preferred applications, the intermediate layer is also a transparent layer, either by virtue of being an inherently transparent material or by virtue of being deposited at a thickness whereby the material is effectively transparent to light of a desired wavelength. The intermediate layer, as shown, also includes an increased ability to bind active molecules of interest as described previously. A subsequent layer 606 is then deposited over the intermediate layer 604 to provide the structure to the aperture(s) 608 and/or provide optical confinement of light entering the aperture 608, e.g., as a cladding layer in a ZMW.

As noted above, for applications of substrates that require optical access through the base substrate into the aperture, the intermediate layer will be either a transparent material, or deposited at a sufficiently small thickness as to be effectively transparent. Metal layers, for example, may be deposited at thicknesses that remain transparent. Gold layers deposited at thicknesses of e.g., from 2 to about 50 nm are generally transparent to most relevant spectra while still providing selective immobilization potential. Other metal layers may likewise be deposited at appropriately thin layers to maintain functional transparency.

Metal layers that are employed as structural or optical confinements, e.g., as a cladding layer for zero mode waveguides will typically be selected for their optical properties. In particular, metals like aluminum, chromium and the like may be employed as cladding layers in accordance with the present invention.

As noted above, the substrates of the invention are particularly useful in the optical analysis of chemical and biochemical reactions. As such, these substrates are typically used in conjunction with optical analysis systems that are positioned to direct light to and receive optical signals from the reactions of interest, e.g., that are occurring in the apertures or openings, e.g., opening 508 in FIG. 5. Such systems typically include an excitation light source, and an appropriate optical train. The light source is positioned to direct light through the optical train at the substrate and particularly the regions of the substrate upon which the reactions of interest are taking place. Optical signals that emanate from the reaction of interest are then passed back through the optical train to an appropriate detector, which will typically include an array detector, such as a diode array detector or a charge coupled device, e.g., a CCD an ICCD or an EMCCD. Additional optical components are typically also included, such as optical filters, dichroics, prisms and mirrors, for the selective direction of light of different wavelengths, e.g., from the excitation source, and/or different signal elements within a reaction of interest. Systems that are particularly useful in conjunction with the substrates of the invention and particularly zero mode waveguide array substrates are described in published U.S. Patent Application No. 2007/0188750, filed Jul. 5, 2006, which is incorporated herein by reference in their entirety for all purposes.

Although described in some detail for purposes of illustration, it will be readily appreciated that a number of variations known or appreciated by those of skill in the art may be practiced within the scope of present invention. To the extent not already expressly incorporated herein, all published references and patent documents referred to in this disclosure are incorporated herein by reference in their entirety for all purposes.

What is claimed:

1. A substrate having active molecules of interest disposed thereon, comprising:
a first layer having a first surface;
a second layer disposed over a portion of the first surface;
wherein the second layer comprises a material having a substantially increased or decreased ability to bind the active molecules of interest, relative to the first layer;
active molecules of interest preferentially bound to one of the first and second layers.

2. The substrate of claim 1, wherein the second layer has a substantially increased ability to bind the molecules of interest relative to the first layer.

3. The substrate of claim 1, wherein the second layer has a substantially increased ability to bind the molecules of interest relative to the first layer.

4. The substrate of claim 2, wherein the molecules of interest are bound to the second layer relative to the first layer at a ratio of at least 5:1.

5. The substrate of claim 2, wherein the molecules of interest are bound to the second layer relative to the first layer at a ratio of at least 10:1.

6. The substrate of claim 2, wherein the molecules of interest are bound to the second layer relative to the first layer at a ratio of at least 50:1.

7. The substrate of claim 2, wherein the molecules of interest are bound to the second layer relative to the first layer at a ratio of at least 100:1.

8. The substrate of claim 1, wherein the second layer has a substantially decreased ability to bind the molecules of interest relative to the first layer.

9. The substrate of claim 2, wherein the molecules of interest are bound to the first layer relative to the second layer at a ratio of at least 5:1.

10. The substrate of claim 2, wherein the molecules of interest are bound to the first layer relative to the second layer at a ratio of at least 10:1.

11. The substrate of claim 2, wherein the molecules of interest are bound to the first layer relative to the second layer at a ratio of at least 50:1.

12. The substrate of claim 2, wherein the molecules of interest are bound to the first layer relative to the second layer at a ratio of at least 100:1.

13. The substrate of claim 1, further comprising a third layer disposed over at least a portion of the second layer.

14. The substrate of claim 1, further comprising a third layer disposed between the portion of the first surface of the first layer and the second layer.

15. The substrate of claim 1, wherein the first layer comprises a transparent layer and the second layer comprises a metal layer.

16. The substrate of claim 15, wherein the metal layer comprises a metal layer having a substantially increased ability to bind the molecules of interest relative to the transparent layer.

17. The substrate of claim 16, wherein the metal layer comprises gold, and the molecules of interest are bound to the second layer through thiol groups.

18. The substrate of claim 1, wherein the second layer comprises an opaque material disposed upon the first surface and further comprising a plurality of apertures disposed through the second layer to the first surface.

19. The substrate of claim 1, wherein the third layer is disposed over the second layer and further comprising a plurality of apertures disposed through the second and third layers to the first surface thereby exposing a portion of the second layer.

20. The substrate of claim 14, wherein the second layer comprises a material having a decreased ability to bind active molecules of interest.

21. The substrate of claim 20, wherein the material of the second layer has a deactivating influence on an activity of the molecules of interest.

22. The substrate of claim 21, wherein the material of the second layer comprises an enzyme having a deactivating influence on the activity of the active molecules of interest.

23. The substrate of claim 21, wherein the material of the second layer comprises TiO₂.

24. The substrate of claim 1, wherein the active molecules of interest comprise nucleic acid polymerase enzymes.

25. A method of selectively providing active molecules of interest in a first location on a substrate, comprising:
providing a first substrate layer having a first surface;
providing a second layer disposed over a portion of the first surface, the second layer having an increased ability to bind the active molecules of interest relative to the first layer;
providing a third layer disposed over at least a portion of the second layer, the third layer having a substantially descreased ability to bind the molecules of interest relative to the second layer, and a portion of the second layer remaining exposed; and
contacting the substrate with the molecules of interest to coupled active molecules of interest preferentially to the portion of the second layer that is exposed, relative to an exposed portion of the first layer and the third layer.

26. A method of analyzing an active molecule of interest, comprising:
providing a layered substrate comprising:
a first transparent layer;
a second layer disposed over the first transparent layer, the second layer having a substantially increased ability to bind the active molecules of interest relative to the first transparent layer;
a third layer disposed over the second layer;
a plurality of apertures disposed through the second and third layers to provide an observation aperture through the transparent layer, an internal wall of the apertures exposing a portion of the second layer; and
active molecules of interest preferentially bound to the portion of the second layer exposed in the apertures;
observing activity of the active molecules of interest through the observation aperture in the transparent substrate.
